(19) United States
(12) Patent Application Publication
(10) Pub. No.: US 2008/0119864 A1
(43) Pub. Date: May 22, 2008

(54) Lens Holder for an Insertion Device for Deformable Intra-Ocular Lenses

(76) Inventors: Klaus Deinzer, Bein-wil am See (CH); Rene Kammerlander, Dietikon (CH)

Correspondence Address: COLLARD & ROE, P.C., 1077 NORTHERN BOULEVARD, ROSLYN, NY, 11576, US

(21) Appl. No.: 10/590,950

(22) PCT Filed: Feb. 27, 2004
(86) PCT No.: PCT/EP04/50225

§ 371 Date: Oct. 26, 2007

(51) Int. Cl. A61F 9/007

(52) U.S. Cl. 606/107

(57) ABSTRACT

The lens holder is used to transfer an intraocular lens from a relaxed state into an elastically deformed state so that it can be injected with the aid of a co-operating device into an eye, where it resumes its relaxed state again. The lens holder (1) contains a flexible backing support (8) which can be deformed from an open position, in which it accommodates an intraocular lens in its relaxed state, into a closed position in which it is inserted in said device. The intraocular lens is inserted in the opened lens holder and the latter is then moved into its closed position by bending it. As a result, the intraocular lens is deformed uniformly and without excessive local stress.

The invention relates to a lens holder for a device for inserting deformable intraocular lenses, by means of which an intraocular lens is transferred from a relaxed state into an elastically deformed state so that it can be injected into an eye, where it then assumes its relaxed state again.

Devices for inserting deformable intraocular lenses are known. Generally speaking, their main purpose is to enable the incision in the eye needed to insert an intraocular lens to be kept as small as possible. A problem which occurs with such devices is that the intraocular lens has to be transferred to an elastically deformed state so that it can be injected through a cannula into an eye in this state. U.S. Pat. No. 4,681,102 discloses a device of this type. In this instance, a lens holder has a hinge, which enables the lens holder to be transferred from an open position, in which the lens is inserted, into a closed state in which the lens is folded. The hinge disposed at the centre of the lens holder initially prevents the lens from deforming and situations may even arise in which the lens flexes at the start of the deformation process and is bent in the direction opposite the intended direction of deformation. The device disclosed in U.S. Pat. No. 5,947,975 offers an improvement in this respect due to the fact that the lens holder has two hinges. Both of the devices mentioned above have a disadvantage due to the fact that it is awkward to position the intraocular lens in the lens holder. Another disadvantage of these devices resides in the fact that in practical terms, they fold the intraocular lens about a line oriented in the longitudinal direction of the device, which can lead to excessive local stress on the lens, causing it to remain permanently deformed under certain circumstances if it is left in the device for too long prior to being injected.

Against the background of this prior art, the underlying objective of the invention is to propose a lens holder of the type outlined above, which enables the intraocular lens to be easily inserted and which enables a uniform deformation of the intraocular lens without causing excessive local stress.

In order to achieve these objectives, the lens holder contains a flexible backing support which can be deformed from an open position, in which it is designed to accommodate an intraocular lens in its relaxed state, into a closed position in which it is designed to be inserted in the device. The expression “flexible” backing support is intended to mean a region which can be elastically, partially plastically or plastically deformed without breaking. As a result of this solution, the intraocular lens is supported during the deformation process so that it can not flex or shift. Furthermore, the intraocular lens is deformed across its entire extension and not just about one or two bending axes as is the case with the prior art. This reliably prevents the intraocular lens from being subjected to excessive local stress.

The lens holder is advantageously designed so that it can be transferred from the open position into the closed position by bending the flexible backing support, whereby an increasing curvature is imparted to the flexible backing support and hence also to the intraocular lens in contact with it. This enables a particularly gentle deformation of the intraocular lens.

In one embodiment, the flexible backing support is elastically deformable between the open position and the closed position so that it resumes its initial position again whenever it is relieved of the deformation force. This being the case, the lens holder may be designed so that it is either relaxed in the open position or so that it is relaxed in the closed position.

In one embodiment of the invention, the flexible backing support forms a passage for accommodating the deformed intraocular lens in the closed position. Said cannula, into which the deformed intraocular lens can be pushed by means of a push rod for example, can be connected to this passage.

In another embodiment, the flexible backing support has two oppositely lying, reinforced peripheral regions. These peripheral regions may fulfil several functions, as will become apparent later on. For example, at the transition from the flexible backing support to the respective peripheral region on either side, an undercut is provided as a means of retaining and guiding the edges of the intraocular lens. These peripheral regions enable the intraocular lens to be accurately positioned in the lens holder and also prevent the intraocular lens from slipping as it is deformed. At least one of the undercut peripheral regions advantageously has a recess so that the edge of the intraocular lens is able to move past the peripheral region of the lens holder unhindered as it is inserted.

A spherical depression may also be provided in the flexible backing support as a means of accommodating the optical part of the intraocular lens, thereby making it easier to position the intraocular lens in the lens holder.

In another embodiment, the flexible backing support has a tapered region at one end between the peripheral regions, forming a guide for a push rod designed to transport the deformed intraocular lens.

In another embodiment, the flexible backing support has a cross-section which constantly varies from the centre outwards towards the two peripheral regions. This enables the lens holder to be deformed in a predefined manner so that the bending line of the intraocular lens can be optimised.

In another embodiment, means are provided in the peripheral regions of the lens holder to provide a mutual connection of the peripheral regions. These connecting means enable the lens holder to be locked in its closed position, which makes it easier to insert the lens holder together with the deformed intraocular lens accommodated in it into a co-operating injection device. In another embodiment, gripping means are provided at the peripheral regions in order to facilitate deformation of the lens holder.

In another embodiment, the passage formed in the closed position becomes narrower towards one end of the lens holder. This enables the lens to be further compressed by pushing it in the passage so that it can finally be transferred to said cannula from which it can be injected into an eye.

In another embodiment of the invention, the transitions from the flexible backing support to the peripheral regions of the passage formed in the closed position are such that the passage has a snail-type cross-section at one of its ends. With a passage of this shape, large intraocular lenses in particular can be rolled so that their oppositely lying edges overlap as they are pushed through the passage.

In another embodiment, at least one of the undercuts becomes larger towards one end of the lens holder in order to form an inlet portion for a haptic disposed on the intraocular lens.

In yet another embodiment, catch means are provided on the lens holder as a means of positioning and retaining it in a housing of said device.

Finally, in another embodiment, the lens holder is made from polypropylene and is preferably manufactured integrally in an injection moulding process.

Examples of embodiments of the invention will be described below with reference to the appended drawings.

Of these:

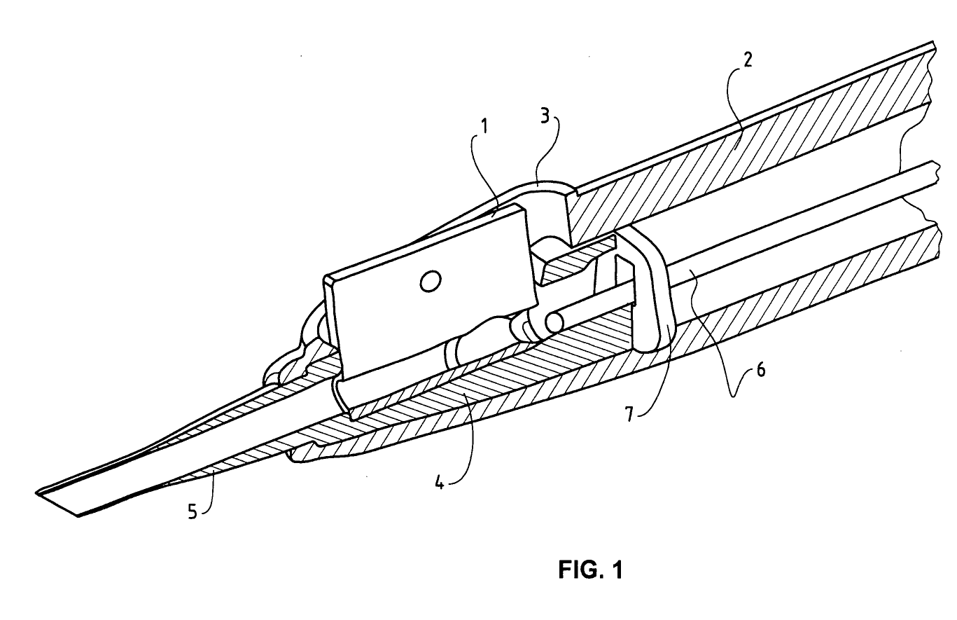

FIG. 1 is a perspective view of a device for inserting deformable intraocular lenses, illustrating one example of an embodiment of a lens holder proposed by the invention,

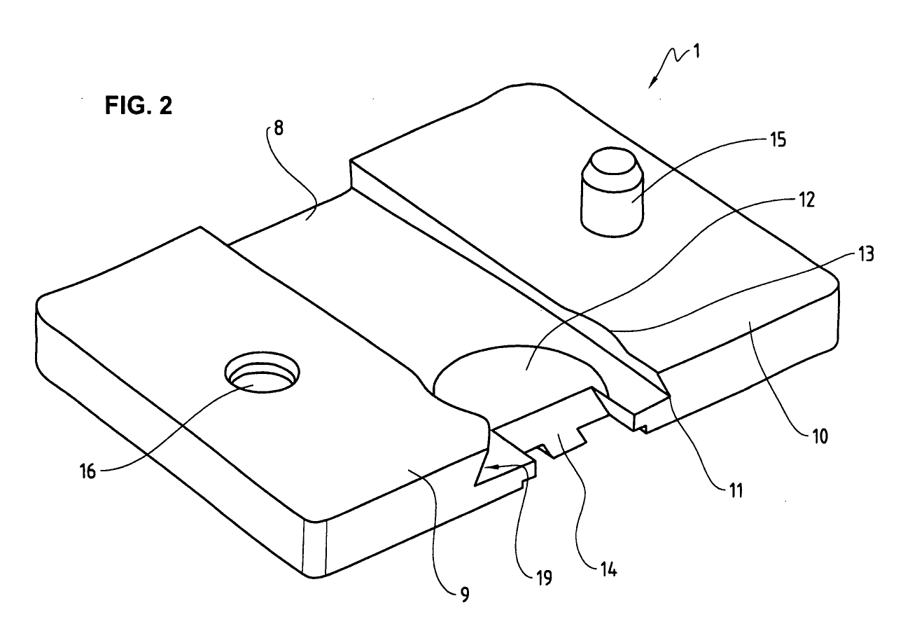

FIG. 2 is a perspective view of a first embodiment of the lens holder in its open position,

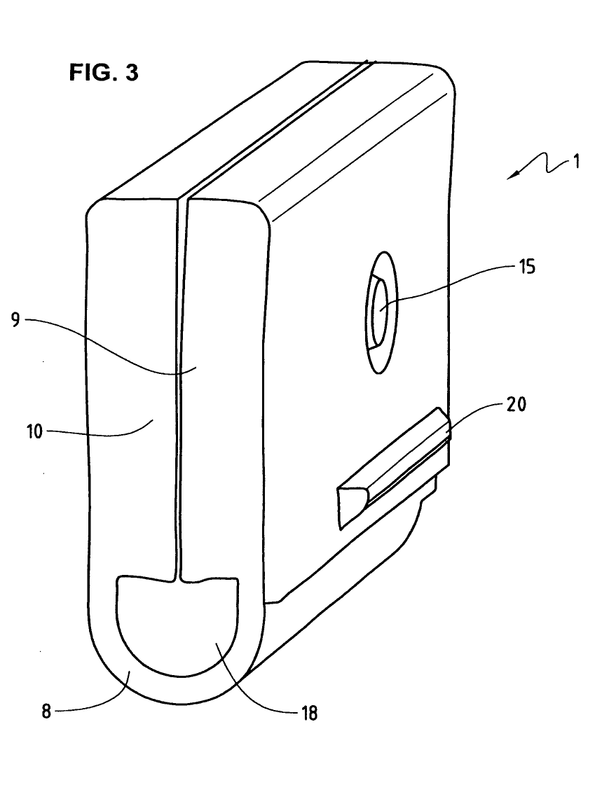

FIG. 3 is a perspective view of the lens holder illustrated in FIG. 2 in its closed position,

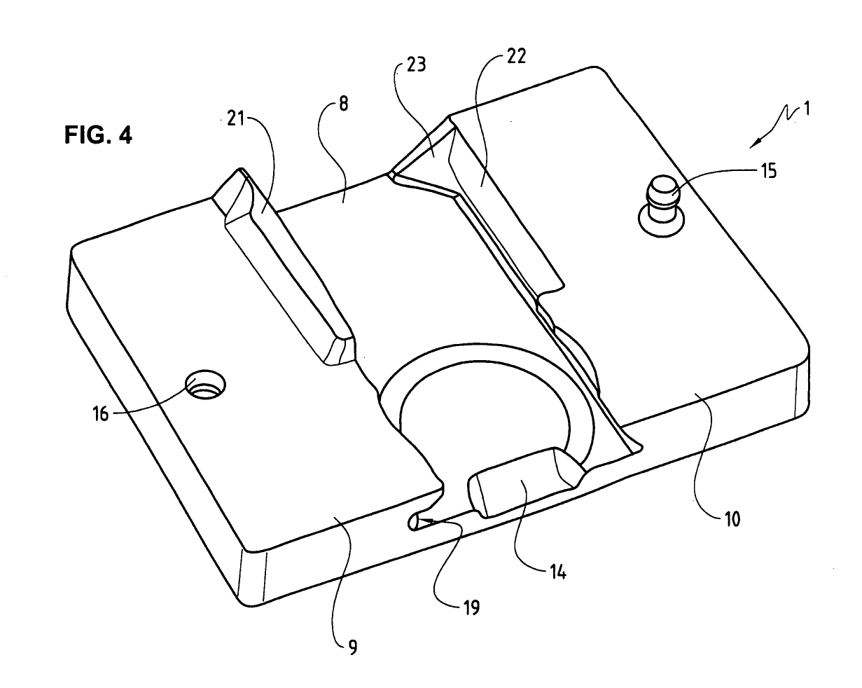

FIG. 4 is a perspective view of a second embodiment of the lens holder in its open position,

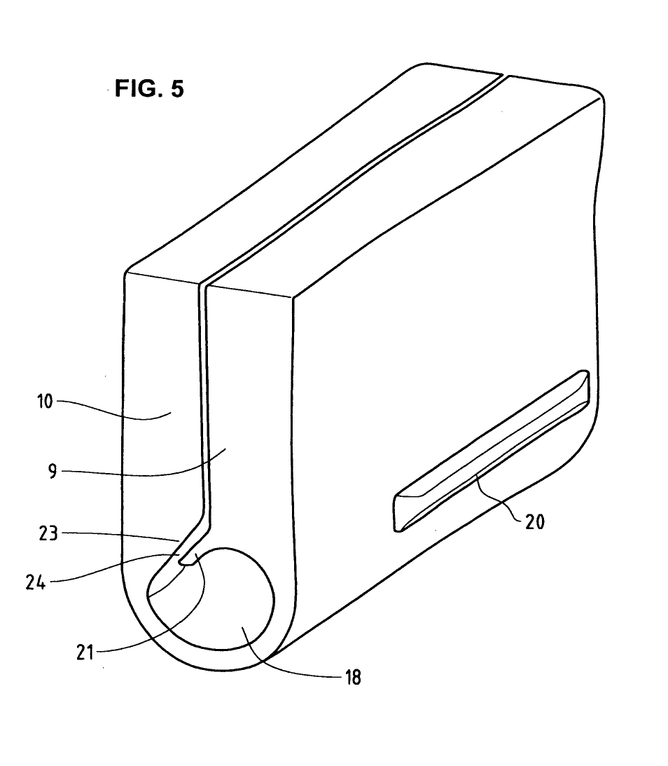

FIG. 5 is a perspective view of the lens holder illustrated in FIG. 4 in its closed position and

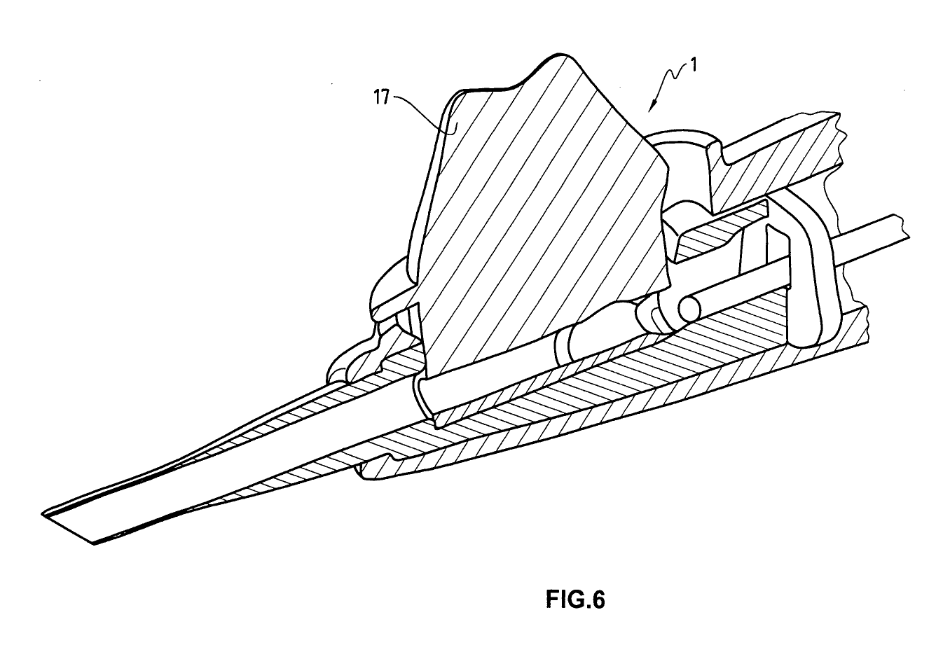

FIG. 6 is a perspective view of a third embodiment of the lens holder in its closed position, fitted in a device.

FIG. 1 shows a detail of a device for inserting deformable intraocular lenses with a lens holder 1 inserted in the device. The device has an elongate housing 2, in the side wall of which an elongate orifice 3 used for inserting the lens holder 1 is provided. In this particular example, the lens holder 1 is retained in a bearing part 4, which bearing part 4 is integrally connected to a cannula 5. When the device is being used, the lens holder 1 with an intraocular lens accommodated it in the deformed state is inserted into the device through the orifice 3. The intraocular lens is then pushed out of the lens holder 1 into the cannula 5 by means of a push rod 6. The cannula 5 is then inserted through a small incision in the eye of a patient and the intraocular lens is pushed out of the cannula 5 into the eye by means of the push rod 6. A guide part 7 disposed in the housing 2 ensures that the push rod 6 moves the intraocular lens precisely.

FIG. 2 shows a perspective view of a first embodiment of the lens holder 1 in its open position in readiness for accommodating an intraocular lens (not illustrated). The lens holder 1 has a flexible backing support 8 which is essentially flat in this position. On either side of the flexible backing support 8, the lens holder 1 has peripheral regions 9 and 10, which are thicker and hence stiffer than the flexible backing support 8. At the transition between the flexible backing support and the peripheral region, an undercut 11 is respectively provided, which enables the edges of the intraocular lens to be inserted to be retained during the deformation operation and guided during displacement into the cannula 5. In order to make it easier to insert the intraocular lens, a respective recess 13 is provided in the two peripheral regions but does not extend as far as the base of the undercut 11. A spherical depression 12 provided in the flexible backing support 8 may further facilitate the positioning of the intraocular lens. Known intraocular lenses have two so-called haptics. These are small pins which centre the lens in the lens bag in the eye. In order to insert such intraocular lenses with the lens holder proposed by the invention, the lens holder 1 advantageously has a widening 19 in the region of one of the undercuts 11, which accommodates a haptic and prevents it from being left caught on the lens holder 1 subsequently when the intraocular lens is being pushed by the push rod 6. Once an intraocular lens has been positioned in the lens holder 1 in the manner described above, it is gripped at its peripheral regions 9 and 10 and the latter is deformed together with the intraocular lens lying on the flexible backing support 8 until it assumes the position illustrated in FIG. 3. A tapered region 14 is provided at one end of the flexible backing support 8, the purpose of which is to guide the push rod 6 after deformation and insertion in the device so that it hits the edge of the deformed intraocular lens precisely when it has to be pushed out of the lens holder 1 into the cannula 5.

FIG. 3 shows a perspective view of the lens holder 1 illustrated in FIG. 2 in its closed position. As clearly illustrated, a passage 18 is bounded by the bent flexible backing support 8 and the peripheral regions 9 and 10. To ensure that the lens holder 1 remains in its closed position illustrated in FIG. 3 until it has been inserted in the device, connecting means are provided which, in this particular example, consist of pins 15 disposed on the peripheral region 10 which engage in orifices 16 provided in the peripheral region 9. Catch means are provided on at least one side of the closed lens holder, for example in the form of a rib 20, designed to position and lock the lens holder 1 in the housing 2 of the device accurately.

FIGS. 4 and 5 illustrate another embodiment of the lens holder 1, which is specially designed for injecting relatively large intraocular lenses. FIG. 4 illustrates the lens holder 1 in the open position and FIG. 5 in the closed position. The same reference numbers are used for parts that are the same as those illustrated in FIGS. 1 to 3. The lens holder 1 illustrated in FIGS. 4 and 5 specifically differs from that illustrated in FIGS. 2 and 3 due to a raised area 21 disposed in the peripheral region 9 adjoining the flexible backing support 8. Provided in the oppositely lying peripheral region 10 is a recess 22, in which the raised area 21 is accommodated when the lens holder 1 is in the closed position. As clearly illustrated in FIG. 5, a snail-shaped cross-section is imparted to the passage 18 at the outlet end due to the raised area 21 and the recess 22. Accordingly, a gap 24 is disposed between the raised area 21 and the recess 22, which enables a large intraocular lens to deform so that its oppositely lying edges overlap, as a result of which the intraocular lens leaves the lens holder 1 in a rolled-up state. In order to transfer the intraocular lens into the cannula 5 rolled up in this manner, a guide part 23 is provided in the peripheral region 10. In this embodiment, the pins 15 are undercut to produce a latching action in the orifices 16.

In the embodiment illustrated in FIG. 6, wing-type grips 17 are provided on the peripheral regions 9 and 10, enabling the relevant lens holder to be more readily gripped, deformed and retained.

In another embodiment of the invention, although this is not illustrated, the flexible backing support may form a loop, which accommodates an intraocular lens in its relaxed state and is then pulled together, for example more or less as happens in the case of cable binders.

1-18. (canceled)

19. Lens holder (1) for a device for inserting deformable intraocular lenses, by means of which an intraocular lens can be transferred from a relaxed state into an elastically deformed state so that it can be injected with the aid of the device into an eye, where it resumes its relaxed state again, and the lens holder (1) contains a flexible backing support (8) with two oppositely lying peripheral regions (9, 10), which flexible backing support (8) can be deformed from an open position, in which it is designed to accommodate an intraocular lens in its relaxed state, into a closed position in which it forms a passage (18) for accommodating the deformed intraocular lens and the lens holder (1) is designed to be inserted in the device in the closed position, characterised in that the transitions from the flexible backing support (8) to the peripheral regions (9, 10) in the passage (18) formed in the closed position are designed so that the passage has a snail-shaped cross-section at one of its ends.

20. Lens holder as claimed in claim 19, wherein it is designed so that it can be transferred from the open into the closed position by bending the flexible backing support (8) and an increasing curvature is imparted to the flexible backing support (8) and hence also to the intraocular lens in contact with it as a result.

21. Lens holder as claimed in claim 19, wherein the flexible backing support (8) can be elastically deformed between the open position and the closed position and is relaxed in the open position.

22. Lens holder as claimed in claim 19, wherein the flexible backing support (8) can be elastically deformed between the open position and the closed position and is relaxed in the closed position.

23. Lens holder as claimed in claim 19, wherein an undercut (11) is provided at the transition from the flexible backing support (8) to the respective peripheral region (9, 10) on either side for retaining and guiding the edges of the intraocular lens.

24. Lens holder as claimed in claim 23, wherein at least one of the undercut peripheral regions (9, 10) has a recess (13) so that when the intraocular lens is inserted, its edge is able to pass the peripheral region of the lens holder unhindered.

25. Lens holder as claimed in claim 19, wherein, a spherical depression (12) is disposed in the flexible backing support in order to accommodate the optical part of the intraocular lens.

26. Lens holder as claimed in claim 19, wherein the flexible backing support (8) has a tapered region (14) at one end between the peripheral regions (9, 10) forming a guide for a push rod (6) for transporting the deformed intraocular lens.

27. Lens holder as claimed in claim 19, wherein the flexible backing support (8) has a cross-section which constantly varies from the centre out towards the two peripheral regions (9, 10).

28. Lens holder as claimed in claim 19, wherein means (15, 16) for mutually connecting the peripheral regions are provided in the peripheral regions (9, 10).

29. Lens holder as claimed in claim 19, wherein gripping means (17) are provided at the peripheral regions to make it easier to deform the flexible backing support (8).

30. Lens holder as claimed in claim 19, wherein the passage (18) formed in the closed position becomes narrower towards one end of the lens holder.

31. Lens holder as claimed in claim 23, wherein at least one of the undercuts (11) becomes larger towards one end of the lens holder in order to form an inlet portion (19) for a haptic disposed in the intraocular lens.

32. Lens holder as claimed in claim 19, wherein it is provided with catch means (20) in order to position and retain the lens holder in a housing (2) of said device.

33. Lens holder as claimed in claim 19, wherein it is made from polypropylene and is preferably manufactured integrally by an injection moulding process.
